(19) United States
(12) Patent Application Publication
(10) Pub. No.: US 2021/0292710 A1
(43) Pub. Date: Sep. 23, 2021

(54) METHOD FOR ENHANCING IMMUNE FUNCTION OF IMMUNE CELL AND IMMUNE CELL LINE PRODUCED USING THE SAME

(71) Applicant: INDUSTRY-ACADEMIC COOPERATION FOUNDATION, YONSEI UNIVERSITY, Seoul (KR)

(72) Inventors: Byoung Chul CHO, Seoul (KR); Kyoung-Ho PYO, Seoul (KR); Chang Gon KIM, Seoul (KR); Sung-Eun KIM, Seoul (KR); Jae Hwan KIM, Seoul (KR); Yeongseon BYEON, Seoul (KR)

(73) Assignee: INDUSTRY-ACADEMIC COOPERATION FOUNDATION, YONSEI UNIVERSITY, Seoul (KR)

(21) Appl. No.: 16/833,011

(22) Filed: Mar. 27, 2020

(30) Foreign Application Priority Data
Mar. 23, 2020 (KR) KR 10-2020-003517

(51) Int. Cl. C12N 5/0783; G01N 33/50

(52) U.S. Cl. CPC C12N 5/0636; G01N 33/5011; C12N 2503/00; C12N 2501/998; C12N 2502/30

(57) ABSTRACT

There is provided a method for enhancing immune function of an immune cell, and an immune cell line produced using the method. A method for enhancing immune function of an immune cell according to an exemplary embodiment of the present invention includes: treating immune cells with a PI3K inhibitor to activate AKT/mTOR downstream signal ex vivo; and culturing the immune cells treated with the PI3K inhibitor such that a lowered AKT level rebounds.

### CROSS-REFERENCE TO RELATED APPLICATIONS

This application claims the priority of Korean Patent Application No. 10-2020-0035171 filed on Mar. 23, 2020, in the Korean Intellectual Property Office, the disclosure of which is incorporated herein by reference.

### BACKGROUND

### Field

The present disclosure relates to a method for enhancing immune function of an immune cell, and an immune cell line produced using the method.

### Description of the Related Art

An immune cell-based therapeutic agent refers to a drug used for activating immune response in a body using immune cells such as dendritic cells, natural killer cells (NK cells), and T cells for treating diseases. In particular, the immune cell-based therapeutic agent has been spotlighted as an anti-cancer therapeutic agent. More specifically, chemotherapy anti-cancer agents and radiation treatment as conventional anti-cancer treatment approaches may cause side effects such as deterioration of immune function, gastrointestinal disorders and hair loss due to death of immune cells in a body together with the death of cancer cells. However, the immune cell-based therapeutic agent may activate the immune cells in the body to attack the cancer cell and thus may minimize the side effects of the above-described treatment approaches.

When using the immune cell-based therapeutic agent, the anti-cancer effect is reduced due to metabolic variation of cancer cells, that is, tumor microenvironment. cancer cells may affect immune cells such as T cells in a variety of ways. More specifically, cancer cells cause the tumor microenvironment, such as depletion of nutrients such as glucose and amino acids, high acidity due to lactic acid accumulation, hypoxia, and upregulation of an immune checkpoint, and thus, inhibit metabolism such as glycolysis of immune cells. The immune cells where glycolysis and other metabolism are inhibited may have deteriorated immune function, that is, anti-cancer effect.

This section “Description of the Related Art” has been set forth to facilitate understanding of the present disclosure. It should not be understood that matters described in this section are recognized as a prior art.

### SUMMARY

Emergence of modified immune cells (engineered T cells) may provide a way to cope with complex diseases such as cancer. In this connection, what is needed in the modified immune cell may be an ability to overcome or reduce immune inhibition by the tumor microenvironment. For example, although immune cells may accurately track and recognize tumor cells, they may not effectively attack cancer when the immune function thereof is deteriorated due to the tumor microenvironment.

PI3K (phosophoinositide 3-kinase) is a lipid and protein catalytic enzyme and may be involved in a signaling system in a cell and may function to phosphorylate a D3 position of an inositol ring of phosphoinositide compounds. PI3K may be classified into three types (Classes I, II, III) based on a structure and a function and contains four p110 catalytic subunits (p110α, p110β, p110δ, p110γ). More specifically, Class I PI3K may be classified into Classes IA and IB again based on a type of subunit regulators involved in activity mechanism of enzyme. Class IA includes PI3Kα, β and δ, and Class IB includes PI3K γ. Class I PI3K phosphorylates PIP2 (phosphatidylinositol-4,5-bisphosphate) to generate PIP3 (phosphatidylinositol-3,4,5-triphosphate) which acts as an important secondary messenger in a signal transduction system. Furthermore, PIP3 activates protein kinase B (AKT), and the activated AKT activates mechanistic target of rapamycin (mTOR). Thus, growth and metabolism of eukaryotic cells may be enhanced via the activity of mTOR.

In this connection, mTOR is a serine/threonine protein kinase belonging to the PI3K-related kinase (PIKK) group and includes mTORC1 (mTOR complex 1) which regulates cell growth and metabolism, and mTORC2 (mTOR complex 2) which regulates cell proliferation and survival. The mTORC1 is a downstream regulator of a PI3K/AKT signaling pathway. In many cancer patients, mTOR1 is overactivated due to genetic mutations such as phosphatase tensin homolog (PTEN) involved in the PI3K/AKT signaling pathway. Thus, in the conventional cancer treatment approach, mTOR1 is inhibited via PI3K inhibition to induce apoptosis of cancer cells.

However, the present inventors have recognized that cancer cells inhibit T cell activity via tumor microenvironment. The cancer cell inhibits mTORC1 in a hypoxic condition, via malnutrition and via other inhibition signals. Furthermore, as mTORC1 is inhibited, surrounding T cells fail to perform anergy, that is, antigen exposure, and have the deteriorate immune function against cancer cells.

Therefore, the present inventors activated mTORC1 via regulation of the PI3K/AKT signaling pathway, thereby enhancing the immune function of T cells.

Conventionally, the PI3K inhibition has been used to induce the death of cancer cells. However, the present inventors have recognized that the PI3K inhibition allows a level of AKT playing a bridge signaling role important for metabolic activity to rebound, and thus the AKT having the increased level becomes a stronger signal to enhance metabolic activity.

Thus, the present inventors have identified that AKT is reduced by inhibiting PI3K inhibition in T cells, mTORC1 is activated via the AKT reduction signal, and the activated mTORC1 stimulates AKT again, thereby increasing the expression of AKT. Furthermore, the present inventors have identified that the increased AKT expression enhances T cell glycolysis, thereby enhancing the metabolic fitness of T cells.

Therefore, the present inventors have come to develop a T cell with enhanced metabolic fitness which may effectively sustain the immune function for a long time under poor tumor microenvironment.

Accordingly, a purpose of the present disclosure is to provide a method for enhancing the immune function of immune cells, the method including treating immune cells with a PI3K inhibitor to activate AKT/mTOR downstream signal ex vivo, and culturing the immune cells treated with the PI3K inhibitor such that a lowered AKT level rebounds.

Furthermore, another purpose of the present disclosure is to provide an immune cell line treated with the PI3K inhibitor using the above-described method, in which a percentage of immune cells expressing GLUT1 among total immune cells of the immune cell line is equal to or greater than 40%.

The purposes of the present disclosure are not limited to those mentioned above. Other purposes not mentioned will be clearly understood by those skilled in the art from the following description.

A first aspect of the present disclosure provides a method for enhancing immune function of an immune cell, the method including: treating immune cells with a PI3K inhibitor to activate AKT/mTOR downstream signal ex vivo; and culturing the immune cells treated with the PI3K inhibitor such that a lowered AKT level rebounds.

In one implementation of the first aspect, the method for enhancing the immune function of immune cells further includes isolating the immune cells from a biological sample, prior to treating the cells with the PI3K inhibitor.

In one implementation of the first aspect, the biological sample includes at least one selected from a group consisting of blood, plasma, lymph node, spleen, thymus, and bone marrow. However, the present disclosure is not limited thereto.

In one implementation of the first aspect, the immune cell includes at least one selected from a group consisting of a dendritic cell, a macrophage cell, a B cell, a T cell, and a NK cell. However, the present disclosure is not limited thereto.

In one implementation of the first aspect, the T cell includes at least one selected from a group consisting of a helper T cell, a cytotoxic T cell, a memory T cell, an effect T cell, a regulatory T cell, a natural killer T cell, a mucosa-related constant T cell, an alpha-beta T cell, and a gamma-delta T cell. However, the present disclosure is not limited thereto.

In one implementation of the first aspect, the T cell contains TCR and/or CAR capable of recognizing an antigen of a target cell; a nucleic acid encoding the TCR and CAR; and a vector containing the nucleic acid.

In one implementation of the first aspect, the NK cell contains TCR and/or CAR capable of recognizing an antigen of a target cell; a nucleic acid encoding the TCR and CAR, and a vector containing the nucleic acid.

In one implementation of the first aspect, the method further includes treating the immune cells with CD3 and CD28 antibodies to activate the immune cell.

In one implementation of the first aspect, the culturing may be carried out for 3 to 4 weeks.

In one implementation of the first aspect, the immune cells are treated with the PI3K inhibitor once or twice a day.

In one implementation of the first aspect, the PI3K inhibitor has a concentration of 0.5 to 1 μM.

In one implementation of the first aspect, the PI3K inhibitor includes at least one selected from a group consisting of PI3Kα, PI3Kβ, PI3Kγ, and PI3Kδ inhibitors. In one implementation of the first aspect, the PI3K inhibitor includes PI3Kδ inhibitor. In this connection, the PI3Kδ inhibitor includes at least one selected from a group consisting of selective PI3Kδ inhibitor 1(compound 7n), idelalisib/CAL-101, GS-1101, IC-87114, ZSTK474, PIK-294, PIK-293, leniolisib/CDZ173, GSK2292767, nemiralisib/GSK2269557, acalisib/GS-9820, PI-3065, AMG319, seletalisib/UCB-5857, umbralisib/TGR-1202, PIK-3, IPI-3063, XL147 analogue, PIK-90, taselisib/GDC0032, SRX3207, pilaralisib/XL147, pictilisib/GDC-0941, AZD8835, LY294002, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34inhibitor 1(compound 19), samotlisib/LY3023414, MTX-211, autophinib, VPS34-IN1, GNE-317, GDC-0084, bimiralisib/PQR309, SAR405, PF-49892416, duvelisib/IPI-145, TG100-115, tenalisib/RP6530, and AZD8186. However, the present disclosure is not limited thereto.

In one implementation of the first aspect, the PI3Kα inhibitor includes at least one selected from a group consisting of XL147 analogue, PIK-90, taselisib/GDC0032, SRX3207, pilaralisib/XL147, alpilisib/BYL719, PIK-71 HCL, A66, gedatolisib/PKI-587, fimepinostat/CUDC-907, CH5132799, serabelisib/TAK-117, 2-D08, HS-173, GDC-0326, GNE-477, pictilisib/GDC-0941, AZD8835, LY294002, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34inhibitor 1(compound 19), samotlisib/LY3023414, MTX-211, autophinib, VPS34-IN1, GNE-317, GDC-0084, bimiralisib/PQR309, SAR405, PF-49892416, BGT226 maleate, and SF2523. However, the present disclosure is not limited thereto.

In one implementation of the first aspect, the PI3Kβ inhibitor includes at least one selected from a group consisting of TGX-221, AZD6482, GSK2636771, LY294002, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34inhibitor 1(compound 19), samotlisib/LY3023414, MTX-211, autophinib, VPS34-IN1, GNE-317, GDC-0084, bimiralisib/PQR309, SAR405, PF-49892416, BGT226 maleate, and AZD8186. However, the present disclosure is not limited thereto.

In one implementation of the first aspect, the PI3Kγ inhibitor includes at least one selected from a group consisting of AS-605240, 3-methyladenine/3-MA, voxtalisib/XL765, AS-252424, AS-604850, CAY10505, CZC24832, IPI-549, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34 inhibitor 1(compound 19), samotlisib/LY3023414, MTX-211, autophinib, VPS34-IN1, GNE-317, GDC-0084, bimiralisib/PQR309, SAR405, PF-49892416, duvelisib/IPI-145, TG100-115, tenalisib/RP6530, BGT226 maleate, and SF2523. However, the present disclosure is not limited thereto.

In one implementation of the first aspect, the method further includes: co-culturing the cultured immune cells together with tumor cells; and evaluating a percentage of an amount of tumor cells killed by the immune cells.

In one implementation of the first aspect, the tumor cell includes at least one selected from a group consisting of lung-cancer, non-small cell lung-cancer, pancreatic cancer, stomach cancer, liver cancer, breast cancer, cervical cancer, thyroid cancer, parathyroid cancer, prostate cancer, gallbladder cancer, biliary tract cancer, non-Hodgkin's lymphoma, Hodgkin's lymphoma, blood cancer, bladder cancer, kidney cancer, ovarian cancer, melanoma, colon cancer, bone cancer, skin cancer, head cancer, uterine cancer, rectal cancer, brain tumor, anal anus cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, urinary tract cancer, renal cell carcinoma, renal pelvic carcinoma, CNS (central nervous system) tumor, primary CNS lymphoma, spinal tumor, brainstem glioma and pituitary adenoma. However, the present disclosure is not limited thereto.

In one implementation of the first aspect, the co-culturing is performed in at least one of normal, hypoxic, and low glucose culture conditions.

In one implementation of the first aspect, in evaluating the percentage of the killed tumor cells, the percentage of the dead tumor cells is in a range of 60 to 65% in the normal culture condition, and is in a range of 45 to 5% in the hypoxic culture condition and is in a range of 35 to 40% in the low glucose culture conditions. However, the present disclosure is not limited thereto and may vary depending on the type of immune cells and the conditions of each environment.

A second aspect of the present disclosure provides an immune cell line, in which the immune cell line is treated with a PI3K inhibitor, in which a percentage of immune cells expressing GLUT1 among total immune cells of the immune cell line is equal to or greater than 40%.

In one implementation of the second aspect, a GLUT1 expression level of the immune cell line may be 2 to 3 times greater than a GLUT1 expression level of a control non-treated with the PI3K inhibitor.

In one implementation of the second aspect, the immune cell line includes at least one selected from a group consisting of a helper T cell, a cytotoxic T cell, a memory T cell, an effect T cell, a regulatory T cell, a natural killer T cell, a mucosa-related constant T cell, an alpha-beta T cell, and a gamma-delta T cell. However, the present disclosure is not limited thereto. In one implementation of the second aspect, when each of the helper T cell and the cytotoxic T cell is treated with the PI3K inhibitor, a lowered AKT level is rebounded to increase to a level 1.5 times or greater than an AKT level before the PI3K inhibitor treatment, for 48 hours. In one implementation of the second aspect, the cytotoxic T cells are treated with a PI3K inhibitor, in which a percentage of cytotoxic T cells expressing GLUT1 among total cytotoxic T cells for the immune cell line is equal to or greater than 80%.

Hereinafter, the present disclosure will be described in more detail through embodiments. However, these embodiments are only intended to illustrate the present disclosure by way of example, and the scope of the present disclosure should not be interpreted as being limited by these embodiments.

The method for enhancing the immune function of the immune cell and the immune cell line produced using the method according to an embodiment of the present disclosure may improve the immune function of the immune cell. More specifically, in the treatment of cancer, more effective treatment may be performed when using the method for enhancing the immune function of the immune cell and the immune cell line produced using the method according to an embodiment of the present disclosure in combination with surgical surgery, radiation therapy, and chemotherapy.

Furthermore, the method for enhancing the immune function of immune cells and the immune cell line produced using the above method not only improve the prognosis, but also increase the function of the immune cells in the body, i.e., viability, to prevent recurrence of the disease.

Furthermore, when applying the method for enhancing the immune function of an immune cell according to an embodiment of the present disclosure to an autoimmune cell, it is possible to improve the function of the immune cell by minimizing side effects such as an immune rejection reaction.

Furthermore, when using the method for enhancing the immune function of an immune cell and the immune cell line produced using the method according to an embodiment of the present disclosure, the immune cell may be maintained and grown for a long time even in the poor tumor microenvironment, thereby to cause successive immune signals for a long time, thereby activating the immune function of more immune cells in vivo.

The effect of the present disclosure is not limited to the effects as exemplified above. More various effects are included herein.

### BRIEF DESCRIPTIONS OF DRAWINGS

The above and other aspects, features and other advantages of the present disclosure will be more clearly understood from the following detailed description taken in conjunction with the accompanying drawings, in which:

FIG. 1 shows metabolism in immune cells caused by a PI3K inhibitor;

FIG. 2 shows a method for identifying cancer cell killing ability of T cells in tumor microenvironment due to PI3K inhibitor treatment;

FIG. 3A to FIG. 3C show results of the cancer cell killing ability of the T cell in tumor microenvironment due to treatment with a PI3K inhibitor;

FIG. 4A shows a pathway for glycolysis. FIG. 4B shows the results of mRNA expression of enzymes involved in T cell glycolysis due to PI3K inhibitor treatment;

FIG. 5A shows the result of the expression level of a GLUT1 protein of the T cell due to PI3K inhibitor treatment. FIG. 5B shows the results of extracellular acidification rate (ECAR) of the T cell due to PI3K inhibitor treatment;

FIG. 6 shows the expression level of pAKT (phosphoAKT) in an active state in T cell due to PI3K inhibitor treatment;

FIG. 7 shows the GLUT1 expression level in T cells due to PI3K inhibitor and AKT inhibitor treatment;

FIG. 8 shows AKT and GLUT1 expression levels in T cells due to PI3K inhibitor and mTORC1 inhibitor treatment;

FIG. 9A shows the results of anti-cancer effects in lung-cancer cell line LLC2 due to PI3K inhibitor treatment. FIG. 9B shows a percentage of immune cells relative to the lung-cancer cell line LLC2 due to PI3K inhibitor treatment;

FIG. 10A shows the results of the anti-cancer effect in the lung-cancer cell line KLN205 due to PI3K inhibitor treatment;

FIG. 10B shows a percentage of immune cells in the lung-cancer cell line KLN205 due to PI3K inhibitor treatment;

FIG. 11 shows a procedure of a method for enhancing immune function of immune cells according to an embodiment of the present disclosure;

FIG. 12 shows the results of GLUT1 expression level in T cell based on a type of a PI3K inhibitor;

FIG. 13 shows the results of the anti-cancer effect by the immune cell line according to an embodiment of the present disclosure; and

FIG. 14 shows infiltration of immune cells into the tumor, that is, the results for the tumor, when the immune cell line according to one embodiment of the present disclosure is used.

### DETAILED DESCRIPTIONS

The advantages and features of the present disclosure, and how to achieve them, will become apparent by referring to the embodiments as described below in detail together with the accompanying drawings. However, the present disclosure is not limited to the embodiments disclosed below, but will be implemented in various different forms. The present embodiments allow the present disclosure to be complete and are provided to completely inform the person of ordinary skill in the technical field to which the present disclosure belongs of the scope of the disclosure. The present disclosure is only defined by the scope of the claims.

As used herein, the term “glucose uptake” means that the mitochondria in the cell absorb glucose which is one of the energy sources required to generate energy ATP.

As used herein, the term “activity and/or activation” may mean an action in which an amount of a substance is changed when being stimulated with various signals or a function is exerted in response to the change in the substance. Furthermore, this activity may allow a signal to be delivered to other substances, thus causing a chain reaction.

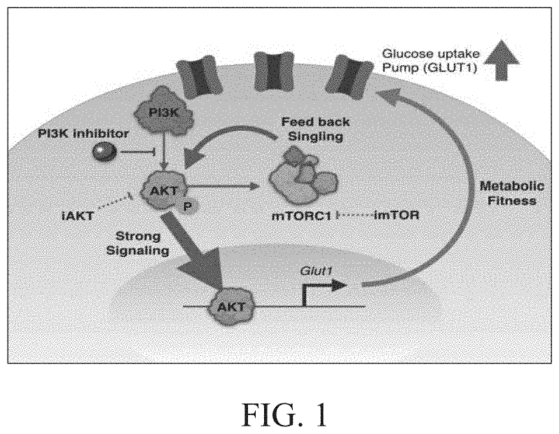

Hereinafter, with reference to FIG. 1, a mechanism of the method for enhancing the immune function of immune cells according to an embodiment of the present disclosure will be described in detail.

Referring to FIG. 1, metabolism in immune cells due to a PI3K inhibitor is shown. Conventionally, T cell immune function enhancement is achieved by inducing AKT/mTOR activity via PI3K activation. Furthermore, the PI3K inhibition has been used to induce the death of cancer cells.

However, unlike the conventional method as described above, the method for enhancing the immune function of the immune cell according to one embodiment of the present disclosure may induce AK/mTOR activity via inhibition of PI3K to enhance the immune function of the immune cell. More specifically, first, the PI3K inhibitor in the immune cell inhibits PI3K present in the cell membrane of the immune cell. Then, the activity of AKT decreases as the PI3K is inhibited.

Then, the signal due to the decrease in activity of AKT stimulates mTORC1, and then the mTORC1 stimulated by the decrease signal may be activated.

Then, the activated mTORC1 as a feedback signal may increase the activity of AKT which has been reduced. In this connection, the feedback signal of mTORC1 becomes a stronger signal than the AKT activity signal increased by the conventional activity of the PI3K, and may cause more enhanced AKT activity.

Then, the activated AKT may increase the expression of GLUT1 and increase glucose uptake into the cell through the GLTU1 receptor present on the cell surface. Thus, the T cell may maintain a competitive nutritional state with the cancer cell in the tumor microenvironment. Accordingly, immune function may be improved via active metabolic activity.

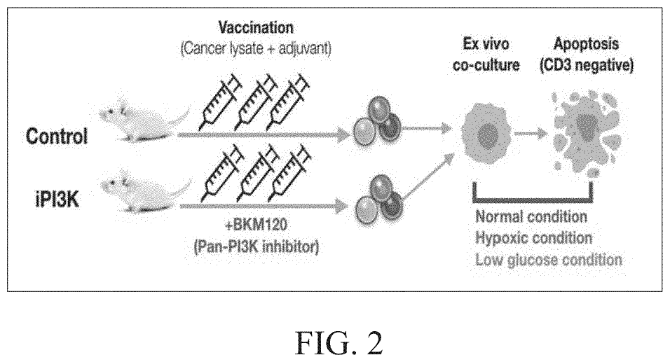

Hereinafter, with reference to FIG. 2 to FIG. 10B, the mechanism for the method for enhancing the immune function of immune cells according to an embodiment of the present disclosure will be identified.

### EXPERIMENTAL EXAMPLE 1

### Identification of Immune Function Enhancement Due to PI3K Inhibitor Treatment

FIG. 2 shows a method for identifying the cancer cell killing ability of T cells in tumor microenvironment due to PI3K inhibitor treatment.

First, 3 μg of a neoplasm lysate obtained from lung-cancer cell line LLC2 and 1 μg of an adjuvant LPS (lipopolysaccharide) are injected into 6 to 8 weeks old BALBc mice as an allogeneic model once a week in an intramuscular injection manner. At the same time, 50 mpk (mg/kg) of BKM-120 as a PI3K inhibitor (iPI3K) is orally administered to the mice belonging to the PI3K inhibitor-treated experimental group once a day.

Then, lymphocytes are separated from the splenocyte of the mouse subjected to the PI3K inhibitor for a total of 3 weeks. Only CD3+ T cells are obtained using a magnetic-activated cell sorter (MACS).

Then, the obtained CD3+ T cells are co-cultured for 24 hours together with the lung-cancer cell line from which the lysate is obtained. In this connection, a ratio between the numbers of the T cell and the lung-cancer cell line may be defined as 10 T cells: 1 lung-cancer cell line. Furthermore, the T cells and lung-cancer cell line are cultured under a normal condition, a hypoxic condition, and a low glucose condition, respectively, to identify cancer cell killing ability in poor tumor microenvironment.

Then, the co-cultured T cells and lung-cancer cell line are subjected to flow cytometry analysis using an apoptosis kit and CD3+ antibodies.

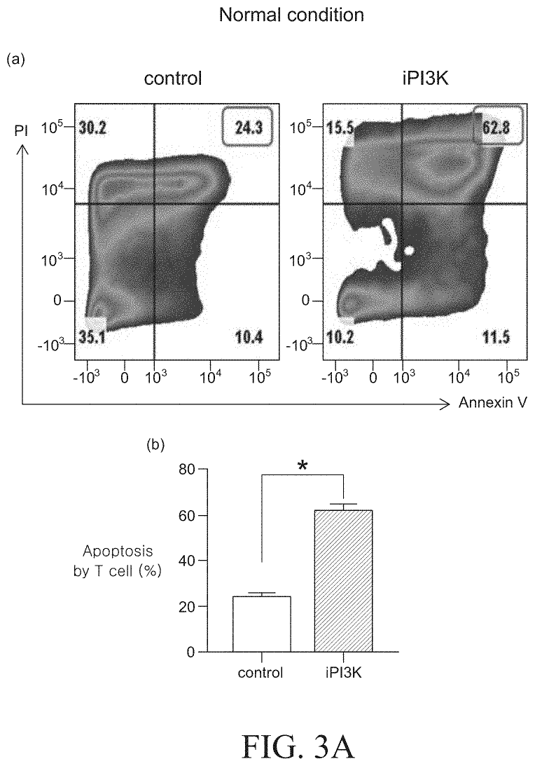

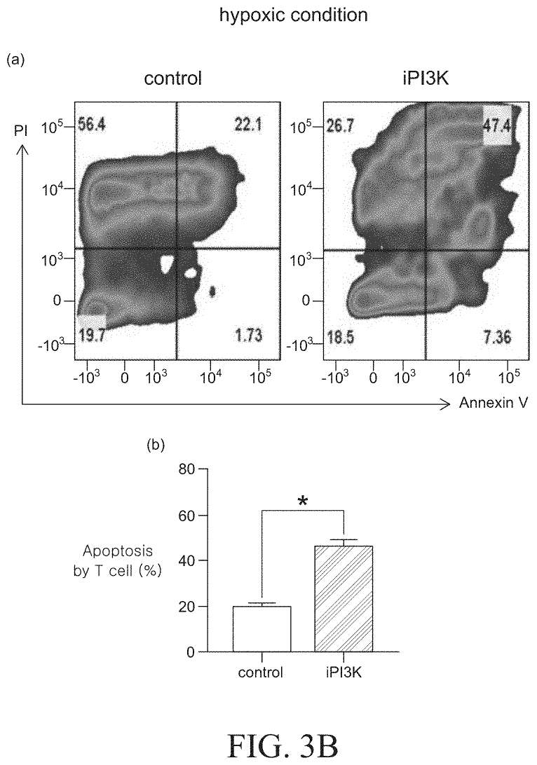

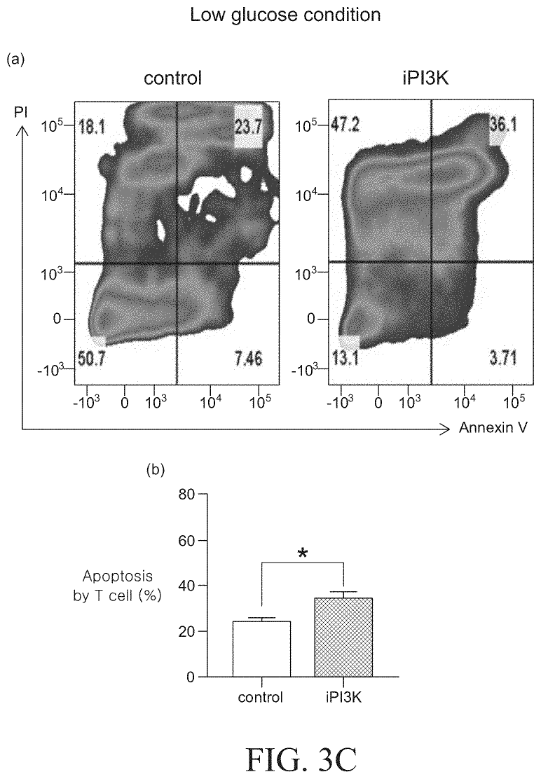

Thus, referring to FIG. 3A to FIG. 3C, the result of the cancer cell killing ability of the T cell in the tumor microenvironment due to the treatment of PI3K inhibitor is shown.

First, referring to (a) of FIG. 3A, the result of the cancer cell killing ability of the T cell under the normal condition due to the treatment of the PI3K inhibitor is shown. In this connection, the x-axis and y-axis indicate apoptosis using annexin V and PI (propidium iodine) as staining reagent, respectively.

A first quadrant of the control is 24.3%, a second quadrant is 30.2%, a third quadrant is 35.1%, and a fourth quadrant is 10.4%. A first quadrant of the PI3K inhibitor-treated group thereof is 62.8%, a second quadrant is 15.5%, a third quadrant is 10.2%, and a fourth quadrant is 11.5%. In this connection, when an entirety of the first quadrant may be stained by both annexin V and PI, this indicates the complete death of the cancer cell. Thus, referring to FIG. 3A (b), which graphically shows the first quadrant of FIG. 3A (a), the treated group treated with the PI3K inhibitor is 62.8%, which is significantly higher than 24.3% of the control (p<0.05). That is, the cancer cell killing ability by the T cell is improved under the normal condition due to PI3K inhibition.

Referring to (a) of FIG. 3B, the result of the cancer cell killing ability by the T cell in the hypoxic condition due to treatment with the PI3K inhibitor is shown.

The first quadrant of the control is 22.1%, the second quadrant is 56.4%, the third quadrant is 19.7%, and the fourth quadrant is 1.73%. The first quadrant of the PI3K inhibitor-treated group is 47.4%, the second quadrant is 26.7%, the third quadrant is 18.5%, and the fourth quadrant is 7.36%. Thus, referring to FIG. 3B (b), which graphically shows the first quadrant of FIG. 3B (a), the treated group treated with the PI3K inhibitor is 47.4%, which is significantly higher than 22.1% of control (p<0.05). That is, it appears that the cancer cell killing ability by the T cell is improved in the hypoxic condition due to PI3K inhibition.

Referring to (a) of FIG. 3C, the result of the cancer cell killing ability by the T cell in the low glucose condition due to the treatment with PI3K inhibitor is shown.

The first quadrant of the control is 23.7%, the second quadrant is 18.1%, the third quadrant is 50.7% and the fourth quadrant is 7.46%. The first quadrant of the PI3K inhibitor-treated group is 36.1%, the second quadrant is 47.2%, the third quadrant is 13.1%, and the fourth quadrant is 3.71%.

Thus, referring to FIG. 3C (b), which graphically shows the first quadrant of FIG. 3C (a), the treated group treated with the PI3K inhibitor is 36.1%, which is significantly higher than 23.7% of the control (p<0.05). That is, the cancer cell killing ability by the T cell is improved in the low glucose condition due to the PI3K inhibition.

### EXPERIMENTAL EXAMPLE 2

### Identification of Glucose Metabolism Enhancement Due to PI3K Inhibitor Treatment

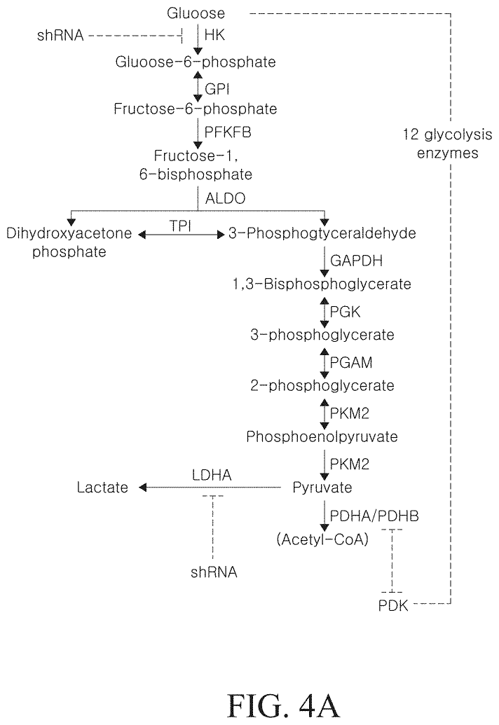

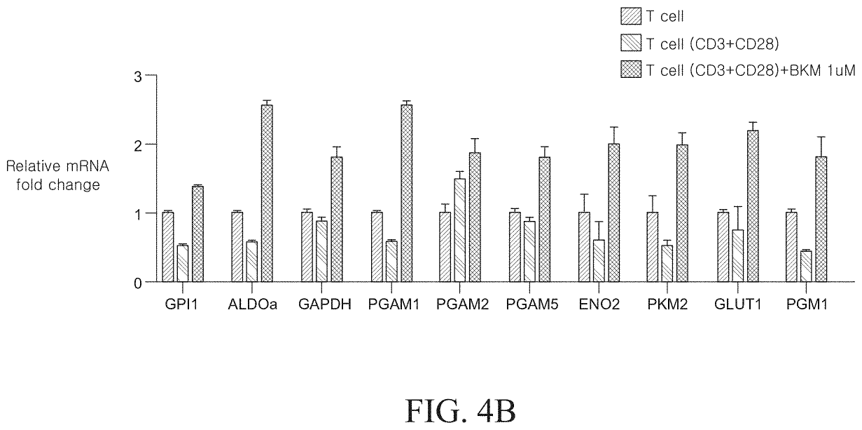

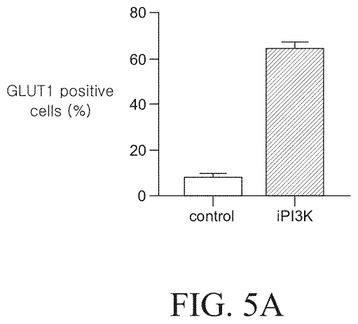

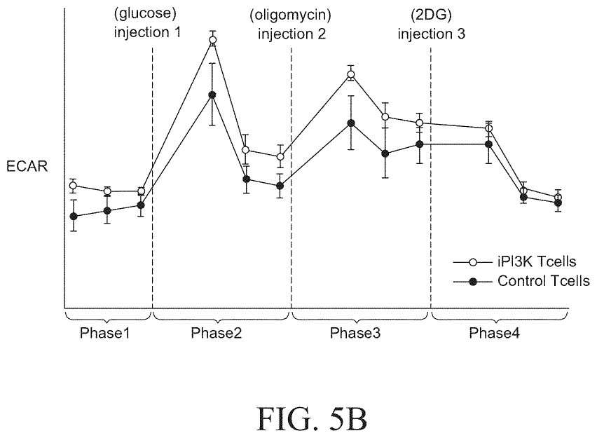

Hereinafter, with reference to FIG. 4A to FIG. 5B, glucose metabolism of T cells due to PI3K inhibitor treatment will be described in detail.

First, referring to FIG. 4A, a pathway for glycolysis is shown. Glycolysis is a key metabolism process in which cells break down glucose to obtain materials necessary for energy and biosynthesis. More specifically, various enzymes such as GPI1, ALDOa, GAPDH, PGAM1, PGAM2, PGAM5, ENO2, PKM2, GLUT1 and PGM1 are involved in the pathway for glycolysis.

Thus, referring to FIG. 4B, the results of mRNA expression of enzymes involved in glycolysis of T cells due to PI3K inhibitor treatment are shown. In this connection, in order to identify enzymes involved in glycolysis of T cells due to PI3K inhibitor treatment, the CD3+ T cells obtained in FIG. 2 are used. Furthermore, the obtained CD3+ T cell is activated for 72 hours using anti-CD3 and anti-CD28 beads to identify mRNA expression of enzymes involved in glycolysis. In this connection, CD3 secretes cytokine as a protein immunomodulator, and CD28 inhibits apoptosis. Only CD3-activated T cells may lead to apoptosis. Therefore, both CD3 and CD28 signals are used to increase T cell function and survival.

The mRNA expression of each enzyme of the T cell group treated with the PI3K inhibitor and stimulated with CD3+ CD28 is higher than that of the T cell separated from spleen cells and that of the T cell group stimulated with CD3+ CD28. That is, the expression of the proteins acting on the glycolysis of T cells is improved due to the PI3K inhibitor treatment.

Furthermore, referring to FIG. 5A, the result of GLUT1 protein expression level of T cell due to PI3K inhibitor treatment is shown. In this connection, T cell is treated in the same method as in FIG. 4B.

In the T cell treated with the PI3K inhibitor, the GLUT1 expression level is significantly 6 to 7 times higher than that of the control. The percentage of T cells expressing GLUT1 among all T cells is in a range of 60 to 70%. In other words, the expression of GLUT1 as a major protein for glucose uptake may increase due to the PI3K inhibitor. Accordingly, glucose uptake of T cells may be improved.

Further, referring to FIG. 5B, the extracellular acidification rate (ECAR) of T cells due to PI3K inhibitor treatment is shown. In this connection, to measure the extracellular acidification rate, the T cell used in FIG. 5A is used, and 1×10⁵ T cells are used. Furthermore, the extracellular acidification rate refers to a measurement of glucose degradation via a change in pH resulting from lactate secretion in the medium.

First, the extracellular acidification rate in phase 1 appears to be higher in the T cell group treated with the PI3K inhibitor than that in the control. In other words, this may mean that glucose degradation, i.e., glycolysis of the T cell is improved due to the PI3K inhibitor treatment.

Then, in both groups, the extracellular acidification rate in phase 2 in which glucose is injected is found to be higher than the extracellular acidification rate in phase 1. In phase 2, the difference between the extracellular acidification rates of the T cell group treated with PI3K inhibitor and the control increases. That is, the improvement of the extracellular acidification rate in phase 2 may mean that the glycolysis of the T cell is increased to use the injected glucose. Furthermore, the T cell group treated with the PI3K inhibitor absorbed more glucose than the control absorbed, due to the increased GLUT1 protein. Thus, the glycolysis is more active, such that the difference increases.

Then, in phase 3 in which oligomycin is injected in order to inhibit the metabolism of mitochondria in the T cell, the extracellular acidification rate which has been decreased is increased again in both groups. Furthermore, the difference between the extracellular acidification rates of the T cell group treated with PI3K inhibitor and the control is similar to that in phase 2. Further, in phase 3, the T cell group treated with PI3K inhibitor showed a higher extracellular acidification rate than that of the control.

In this connection, as the oligomycin is injected to inhibit mitochondrial metabolism, T cells must produce ATP using only glycolysis. Thus, the extracellular acidification rate in phase 3 may mean pure glycolysis capacity.

Therefore, the production amount of ATP via glycolysis is larger in the T cell group treated with PI3K inhibitor than that in the control. This may further activate the immune function of the T cell.

Then, in phase 4 in which 2-deoxy-D-glucose (2DG) is injected, both groups showed decrease in the extracellular acidification rate compared to phase 3. However, the T cell group treated with PI3K inhibitor showed a higher extracellular acidification rate than that in the control.

In this connection, 2DG is a molecule in which the 2-hydroxyl group of glucose is replaced with hydrogen and thus glucose may not be degraded. Furthermore, when the T cell is treated with 2DG, the extracellular acdification rate may be controlled in an upstream phase of glucose metabolism. Thus, the extracellular acidification rate in phase 4 may refer to the glucose reserve in the cell.

Therefore, the amount of glucose reserved in the T cell is larger in the T cell group treated with PI3K inhibitor than that in the control. Thus, the T cell may maintain the immune function for a longer time duration in poor conditions in which no more nutrients are supplied thereto.

Referring to the Experimental Example 2 as described above, immune cells have increased expression levels of various proteins involved in glycolysis such as GPI1, ALDOa, GAPDH, PGAM1, PGAM2, PGAM5, ENO2, PKM2, GLUT1 and PGM1, due to the treatment of PI3K inhibitor thereto. Thus, metabolism for glycolysis increases. Furthermore, immune cells may produce more ATP via improved glycolysis due to treatment with the PI3K inhibitor. Thus, the reserve amount of glucose in the immune cells may be improved. Accordingly, activation of the immune function of the T cell may be improved, and the immune function may be maintained for a long time even in a poor condition in which energy is not easily supplied.

### EXPERIMENTAL EXAMPLE 3

### Identification of AKT Activation Due to PI3K Inhibitor Treatment

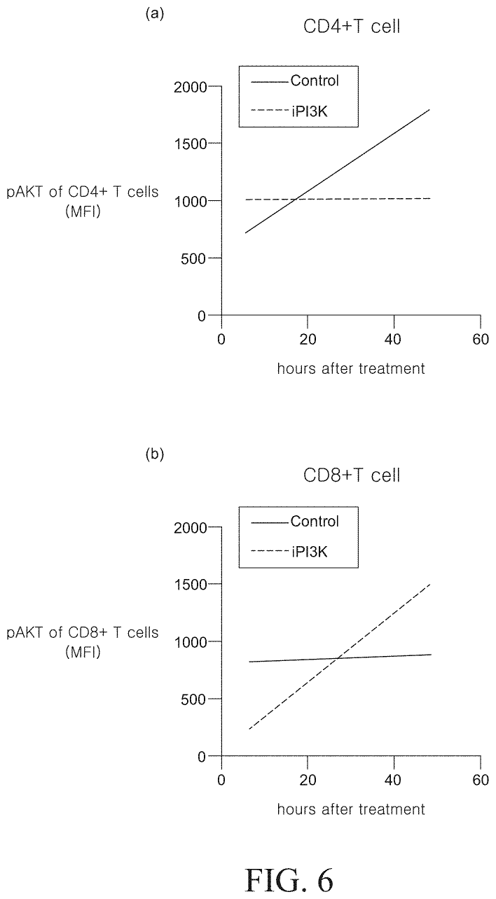

Hereinafter, AKT activation of T cells due to PI3K inhibitor treatment will be described in detail with reference to FIG. 6 to FIG. 8.

First, referring to FIG. 6, an expression level of pAKT (phosphoAKT) in an active state in the T cell due to PI3K inhibitor treatment is shown. In this connection, in order to identify AKT activation of T cells due to PI3K inhibitor treatment, the CD3+ T cells obtained in FIG. 2 are used. Furthermore, the obtained CD3+ T cells are co-cultured for 48 hours together with CD11c positive dendritic cells, anti-CD3 and anti-CD28. The pAKT expression is measured via flow cytometry in 6 and 48 hours after the co-culture has started. Furthermore, expression of pAKT is indicated as mean fluorescence intensity (MFI). Furthermore, CD4 and CD8 positive cells are used as T cells to measure expression of pAKT. In this connection, CD4 and CD8 are present in the cell membrane of the T cell and are cell-binding proteins that bind to non-polymorphic regions of class II MHC and class I MHC, respectively. CD4 may be a helper T cell as a T cell lineage marker. CD8 may refer to a cytotoxic T cell.

More specifically, referring to (a) of FIG. 6, the expression change of AKT in the helper T cell due to PI3K inhibitor treatment is shown. The PI3K inhibitor-treated group has lower AKT expression than that of the control for 0 to 20 hours. However, after 20 hours, the PI3K inhibitor-treated group has higher AKT expression than that of the control. Furthermore, after 48 hours, the AKT expression of the PI3K inhibitor-treated group is 1.5 to 2 times higher than that of the control.

As a result, AKT expression of the helper T cell due to the PI3K inhibitor increases over time. That is, PI3K inhibition in the helper T cells may rebound the expression of AKT.

Referring to (b) of FIG. 6, the expression change of AKT in the cytotoxic T cells due to PI3K inhibitor treatment is shown. The PI3K inhibitor-treated group has lower AKT expression than that of the control for 0 to 25 hours. However, after 25 hours, the PI3K inhibitor-treated group has higher AKT expression than that of the control. Furthermore, after 48 hours, the AKT expression of the PI3K inhibitor-treated group is 1.3 to 1.7 times higher than that of the control.

That is, the AKT expression of the cytotoxic T cell due to the PI3K inhibitor increases over time. That is, PI3K inhibition in the cytotoxic T cells may rebound the expression of AKT.

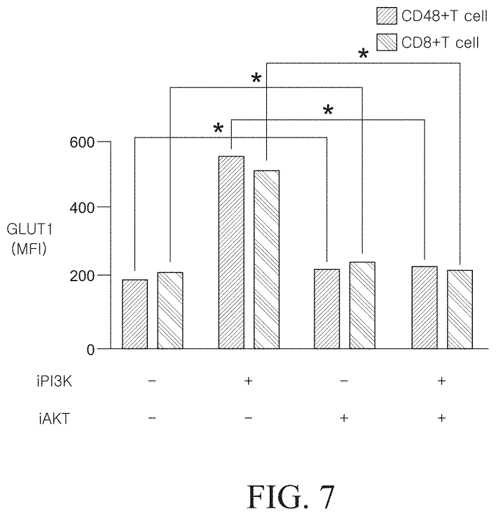

Furthermore, referring to FIG. 7, the GLUT1 expression level in the T cells due to PI3K inhibitor and AKT inhibitor treatment is shown. More specifically, in both the helper and cytotoxic T cells, GLUT1 expression is significantly 2 to 3 times higher in only PI3K inhibitor-treated group (iPI3K+/iAKT−) than those in PI3K and AKT inhibitors non-treated group (iPI3K−/iAKT−), only AKT inhibitor-treated group (iPI3K−/iAKT+), and PI3K and AKT inhibitors-treated group (iPI3K+/iAKT+) (p<0.05).

Furthermore, there is no statistical difference between the expressions of GLUT1 in the PI3K and AKT inhibitors non-treated group (iPI3K−/iAKT−), only AKT inhibitor-treated group (iPI3K−/iAKT+), and PI3K and AKT inhibitors-treated group (iPI3K+/iAKT+).

That is, the activity of AKT may be improved via PI3K inhibition, and the activated AKT may improve the expression of GLUT1 as a T cell glucose receptor. Furthermore, the improvement of the expression of GLUT1 in the T cell may occur only via PI3K inhibition. Direct inhibition of AKT via the inhibitor does not affect the expression of GLUT1.

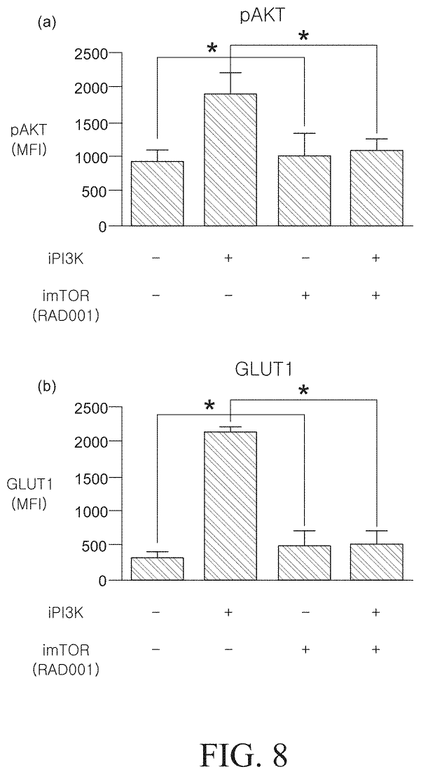

Furthermore, referring to FIG. 8, AKT and GLUT1 expression levels in T cells due to PI3K inhibitor and mTORC1 inhibitor treatment are shown. In this connection, RAD001 is used as a mTORC1 inhibitor.

First, referring to (a) of FIG. 8, AKT expression levels in T cells due to PI3K inhibitor and mTORC1 inhibitor treatment are shown. More specifically, AKT expression is significantly 1.5 to 2.3 times higher (p<0.05) in the only PI3K inhibitor-treated group (iPI3K+/imTOR−) than those in the PI3K and AKT inhibitors non-treated group (iPI3K−/imTOR−), the group treated with only mTORC1 inhibitor (iPI3K−/imTOR+), and the group treated with PI3K and mTORC1 inhibitors (iPI3K+/imTOR+). This may mean that when mTORC1 is inhibited, AKT expression is reduced, and thus mTORC1 is an important signal capable of rebounding AKT expression.

Furthermore, referring to (b) of FIG. 8, the GLUT1 expression level in T cells due to PI3K inhibitor and mTORC1 inhibitor treatment is shown. More specifically, the GLUT1 expression is significantly 6 to 7 times higher (p<0.05) in the only PI3K inhibitor-treated group (iPI3K+/imTOR−) than those in the PI3K and AKT inhibitors non-treated group (iPI3K−/imTOR−), the only mTORC1 inhibitor-treated group (iPI3K−/imTOR+), and the PI3K and mTORC1 inhibitors-treated group (iPI3K+/imTOR+).

In other words, the activity of mTORC1 may be enhanced via PI3K inhibitor, and the activated mTORC1 may activate AKT to improve the expression of GLUT1.

According to Experimental Example 3 described above, the conventional treatment of PI3K inhibitors toward normal cells and cancer cells reduces the activity of the PI3K/AKT/mTOR signaling pathway, leading to apoptosis. However, PI3K inhibitor treatment to T cells may activate the PI3K/AKT/mTOR signaling pathway using a stronger signal. More specifically, due to the conventional PI3K inhibitor treatment toward normal cells and cancer cells, AKT activity is reduced, and thus, all of downstream signals are reduced, resulting in apoptosis. However, AKT activity in the T cell as an immune cell is reduced due to the PI3K inhibitor treatment, and the signal due to the decrease in AKT activity may activate mTORC1. Furthermore, the activated mTORC1 may act as a feedback signal to re-stimulate AKT whose activity level is reduced, thereby to increase AKT activity. Thus, AKT expression decreases for 0 to 25 hours since the PI3K inhibitor treatment, but in 20 hours since PI3K inhibitor treatment, AKT is re-stimulated and AKT expression rebounds due to a stronger signal. Accordingly, the expression of GLUT1 may be heterogeneously increased due to the increase in AKT expression.

### EXPERIMENTAL EXAMPLE 4

### Identification of Anti-Cancer Effects Due to PI3K Inhibitor Treatment

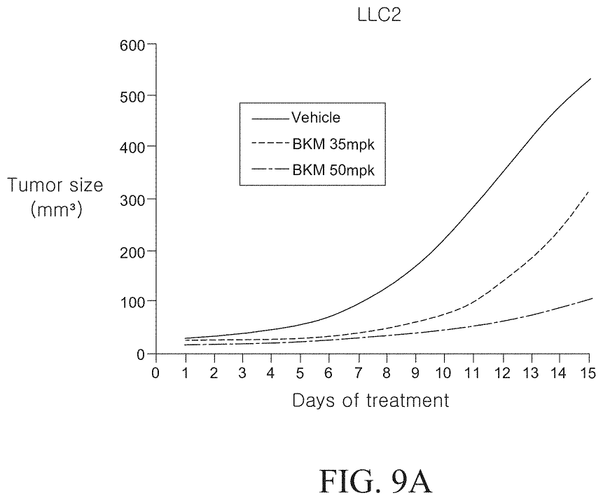

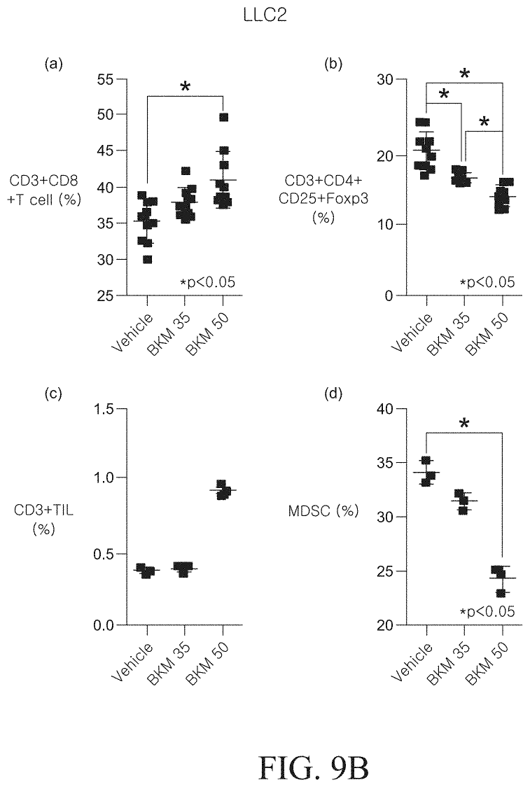

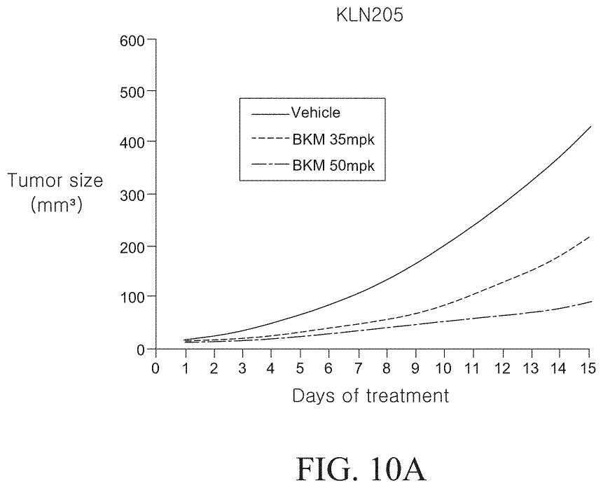

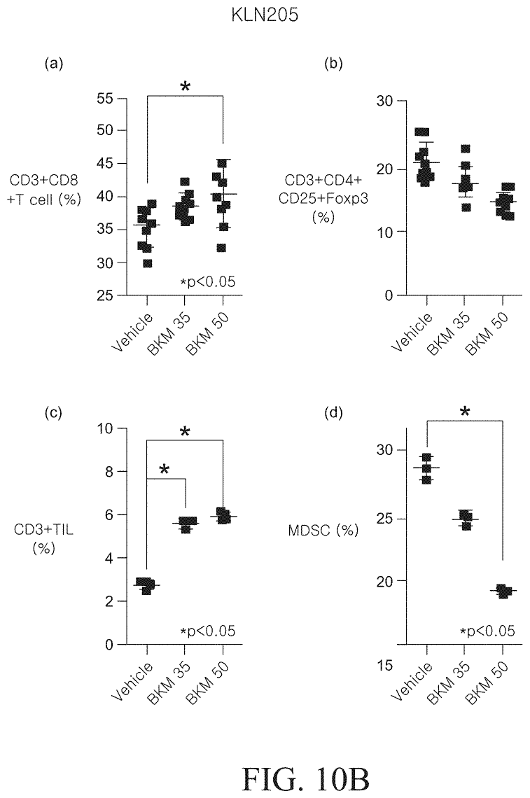

Hereinafter, the anti-cancer effect in the lung-cancer cell line due to PI3K inhibitor treatment will be described in detail with reference to FIG. 9A to FIG. 10B.

First, referring to FIG. 9A, the results of the anti-cancer effect in the lung-cancer cell line LLC2 due to PI3K inhibitor treatment are shown. When the cell line is treated with a vehicle as a control, the tumor size rapidly increased as the days of treatment increased, and the tumor size on the 15th day is 500 to 550 mm³. However, when the PI3K inhibitor treatment is carried out, the tumor size is reduced compared to the control. More specifically, when the PI3K inhibitor BKM 120 treatment is carried out at 35 mpk, the tumor size on the 15th day is 300 to 350 mm³. When BKM 120 treatment is carried out at 50 mpk, the tumor size on day 15 is found to be 90 to 100 mm³.

That is, as the amount of the PI3K inhibitor increases, the tumor size decreases. Thus, the anti-cancer effect increases proportionally to the dose amount of the PI3K inhibitor.

Referring to FIG. 9B, the percentage of immune cells relative to the lung-cancer cell line LLC2 due to PI3K inhibitor treatment is shown.

Referring first to FIG. 9B (a), the percentage of CD3+ CD8+ T cells relative to LLC2 as a lung-cancer cell line due to PI3K inhibitor treatment is shown. In this connection, CD3+ CD8+ T cells may refer to cytotoxic T cells (Tc cells) capable of destroying damaged cells, infected cells, and tumor cells.

When the cell is treated with the vehicle, the distribution of percentages of CD3+ CD8+ T cells is in a range of 30 to 40% and has an average percentage of 35%. Furthermore, when the cell is treated with BKM 120 as a PI3K inhibitor at 35 mpk, the distribution of percentages of CD3+ CD8+ T cells is in a range of 35 to 42% and has an average percentage of 38%. When the cell is treated with BKM 120 at 50 mpk, the distribution of percentages of CD3+ CD8+ T cells is in a range of 37 to 50% and has an average percentage of 42%.

Furthermore, the average percentage thereof when the cell is treated with BKM 120 at 50 mpk is significantly higher than the average percentage when the cell is treated with the vehicle (p<0.05).

That is, as the dose of the PI3K inhibitor increases, the percentage of the CD3+ CD8+ T cell that may increase the anti-cancer effect increases. Thus, the anti-cancer effect increases proportionally to the dose amount of the PI3K inhibitor.

Next, referring to (b) of FIG. 9B, the percentage of CD3+ CD4+ CD25+ Foxp3 T cells relative to LLC2 as a lung-cancer cell line due to the PI3K inhibitor treatment is shown. In this connection, the CD3+ CD4+ CD25+ Foxp3 T cells refer to regulatory T cells (Treg) that may maintain immuno-tolerance and autoimmunity, that is, may inhibit the immune response of other cells.

When the cell is treated with the vehicle, the distribution of percentage of CD3+ CD4+ CD25+ Foxp3 T cells is in a range of 17 to 26% and has an average percentage of 21%. Furthermore, when the cell is treated with BKM 120 as a PI3K inhibitor at 35 mpk, the distribution of percentage of CD3+ CD4+ CD25+ Foxp3 T cells is in a range of 16 to 19%, with an average of 17%. When the cell is treated with BKM 120 at 50 mpk, the distribution of percentage of CD3+ CD4+ CD25+ Foxp3 T cells is in a range of 13 to 17% and has an average of 15%.

Furthermore, the average percentage when the cell is treated with BKM 120 at 35 mpk is significantly lower than the average percentage when the cell is treated with the vehicle (p<0.05). The average percentage when the cell is treated with BKM 120 at 50 mpk is significantly lower than the average percentage when the cell is treated with BKM 120 at 35 mpk (p<0.05).

That is, as the dose of PI3K inhibitor increases, the percentage of CD3+ CD4+ CD25+ Foxp3 T cells which may reduce the anti-cancer effect of immune cells decreases. Thus, the anti-cancer effect increases proportionally to the dose of the PI3K inhibitor.

Next, referring to (c) of FIG. 9B, the percentage of CD3+ TIL cells relative to LLC2 as a lung-cancer cell line due to the PI3K inhibitor treatment is shown. In this connection, CD3+ TIL cell may refer to a tumor infiltrating lymphocyte (TIL) that infiltrates a tumor cell cluster, thereby enhancing the T cell's tumor killing ability, that is, an anti-cancer effect of the T cell.

When the cell is treated with the vehicle, the percentage of CD3+ TIL cells is found to be 0.4% on average. Furthermore, when the cell is treated with PI3K inhibitor BKM 120 at 35 mpk, the percentage of CD3+ TIL cells is found to be 0.4% on average. When the cell is treated with BKM 120 at 50 mpk, the percentage of CD3+ TIL cells is found to be 0.9% on average.

That is, as the dose of the PI3K inhibitor increases, the percentage of the CD3+ TIL T cell which may increase the anti-cancer effect increases. Thus, the anti-cancer effect increases proportionally to the dose of the PI3K inhibitor.

Next, referring to (d) of FIG. 9B, the percentage of MDSC cells relative to the lung-cancer cell line LLC2 due to the PI3K inhibitor treatment is shown. In this connection, the MDSC cell may refer to immature myeloid cells which inhibit the immune response of immune cells such as T cells and NK cells, and induce the generation of regulatory T cells (Treg), thereby to promote the growth of cancer cells.

When the cell is treated with the vehicle, the distribution of percentages of the MDSC cells is in a range of 33 to 35%, with an average percentage of 34%. Furthermore, when the cell is treated with BKM 120 as a PI3K inhibitor at 35 mpk, the distribution of the percentage of MDSC cells is in a range of 31 to 33% and has an average percentage of 32%. When the cell is treated with BKM 120 at 50 mpk, the distribution of the percentage of MDSC cells is in a range of 22 to 26% and has an average percentage of 24%.

Furthermore, the average percentage when the cell is treated with BKM 120 at 50 mpk is significantly lower than the average percentage when the cell is treated with the vehicle (p<0.05).

That is, as the dose of PI3K inhibitor increases, the percentage of MDSC cells capable of reducing the anti-cancer effect of immune cells decreases. Thus, the anti-cancer effect increases proportionally to the dose of the PI3K inhibitor.

Referring to FIG. 10A, the results of the anti-cancer effect against the lung-cancer cell line KLN205 due to the PI3K inhibitor treatment are shown. When the cell is treated with the vehicle as a control, the tumor size rapidly increases as the treatment days increase, and then the tumor size on the 15th day is 400 to 500 mm³. However, when the cell is treated with the PI3K inhibitor, the tumor size is reduced compared to the control. More specifically, when the cell is treated with the PI3K inhibitor BKM 120 at 35 mpk, the tumor size on the 15th day is 200 to 250 mm³. When the cell is treated with BKM 120 at 50 mpk, the tumor size on day 15 is found to be 90 to 100 mm³.

That is, as the dose of the PI3K inhibitor increases, the tumor size decreases. Thus, the anti-cancer effect increases proportionally to the dose of the PI3K inhibitor.

Referring to FIG. 10B, the percentage of immune cells relative to the lung-cancer cell line KLN205 due to PI3K inhibitor treatment is shown.

Referring first to FIG. 10B (a), the percentage of CD3+ CD8+ T cells relative to the lung-cancer cell line KLN205 due to PI3K inhibitor treatment is shown.

When the cell is treated with the vehicle, the distribution of the percentage of CD3+ CD8+ T cells is in a range of 30 to 40% and has an average percentage of 35%. Furthermore, when the cell is treated with BKM 120 as a PI3K inhibitor at 35 mpk, the distribution of the percentage of CD3+ CD8+ T cells is in a range of 35 to 49% and has an average percentage of 39%. When the cell is treated with BKM 120 at 50 mpk, the distribution of the percentage of CD3+ CD8+ T cells is in a range of 32 to 50% and has an average percentage of 41%.

Furthermore, the average percentage when the cell is treated with BKM 120 at 50 mpk is significantly higher than the average percentage when the cell is treated with the vehicle (p<0.05). That is, as the dose of the PI3K inhibitor increases, the percentage of the CD3+ CD8+ T cell that may increase the anti-cancer effect increases. Thus, the anti-cancer effect increases proportionally to the dose of the PI3K inhibitor.

Next, referring to (b) of FIG. 10B, the percentage of CD3+ CD4+ CD25+ Foxp3 T cells relative to lung-cancer cell line KLN205 due to PI3K inhibitor treatment is shown.

When the cell is treated with the vehicle, the distribution of the percentage of CD3+ CD4+ CD25+ Foxp3 T cells is in a range 15 to 26% and has an average percentage of 21%. Furthermore, when the cell is treated with the PI3K inhibitor BKM 120 at 35 mpk, the distribution of the percentage of CD3+ CD4+ CD25+ Foxp3 T cells is in a range of 13 to 24% and has an average of 17%. When the cell is treated with BKM 120 at 50 mpk, the distribution of the percentage of CD3+ CD4+ CD25+ Foxp3 T cells is in a range of 12 to 17% and has an average of 15%.

That is, as the dose of PI3K inhibitor increases, the percentage of CD3+ CD4+ CD25+ Foxp3 T cells, which may reduce the anti-cancer effect of immune cells, decreases. Thus, the anti-cancer effect increases proportionally to the dose of the PI3K inhibitor.

Next, referring to (c) of FIG. 10B, the percentage of CD3+ TIL cells relative to the lung-cancer cell line KLN205 due to PI3K inhibitor treatment is shown.

When the cell is treated with the vehicle, the percentage for CD3+ TIL cells is found to be 3% on average. Furthermore, when the cell is treated with PI3K inhibitor BKM 120 at 35 mpk, the percentage of CD3+ TIL cells is found to be 5% on average. When the cell is treated with BKM 120 at 50 mpk, the percentage of CD3+ TIL cells is found to be 6% on average.

Furthermore, the average percentages when the cell is treated with BKM 120 at 35 mpk and 50 mpk are significantly higher than that when the cell is treated with the vehicle (p<0.05).

That is, as the dose of the PI3K inhibitor increases, the percentage of the CD3+ TIL T cell, which may increase the anti-cancer effect increases. Thus, the anti-cancer effect increases proportionally to the dose of the PI3K inhibitor.

Next, referring to (d) of FIG. 10B, the percentage of MDSC cells relative to lung-cancer cell line KLN205 due to PI3K inhibitor treatment is shown.

When the cell is treated with the vehicle, the distribution of the percentage of MDSC cells is in a range of 26 to 28% and has an average percentage of 27%. Furthermore, when the cell is treated with the BKM 120 as a PI3K inhibitor at 35 mpk, the distribution of the percentage of MDSC cells is in a range of 23 to 25%, with an average of 24%. When the cell is treated with BKM 120 at 50 mpk, the distribution of the percentage of MDSC cells is in a range of 17 to 19% and has an average percentage of 18%.

Furthermore, the average percentage when the cell is treated with BKM 120 at 50 mpk is significantly lower than the average percentage when the cell is treated with the vehicle (p<0.05).

That is, as the dose of PI3K inhibitor increases, the percentage of MDSC cells capable of reducing the anti-cancer effect of immune cells decreases. Thus, the anti-cancer effect increases proportionally to the dose of the PI3K inhibitor.

According to Experimental Example 4 described above, the percentage of cells with cytotoxic and anti-cancer functions of immune cells may increase due to the treatment of the PI3K inhibitor. The percentage of cells having a function of inhibiting the cytotoxic and anti-cancer functions of immune cells may be reduced due to the treatment of the PI3K inhibitor. That is, as the percentage of immune cells inhibiting immune function decreases, cells having cytotoxic and anti-cancer functions are more activated, and thus the anti-cancer effect may be further improved. This may further improve the immune response of immune cells against damaged cells, infected cells, and tumor cells via the PI3K inhibitor.

### EXAMPLE 1

### Method for Enhancing Immune Function of Immune Cell and Immune Cell Line Produced Using the Method

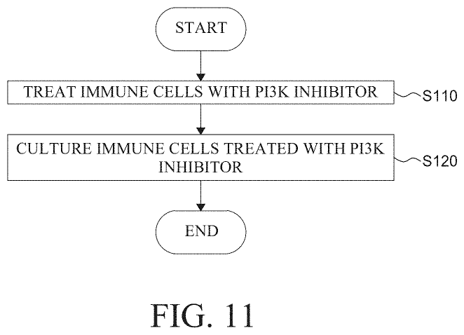

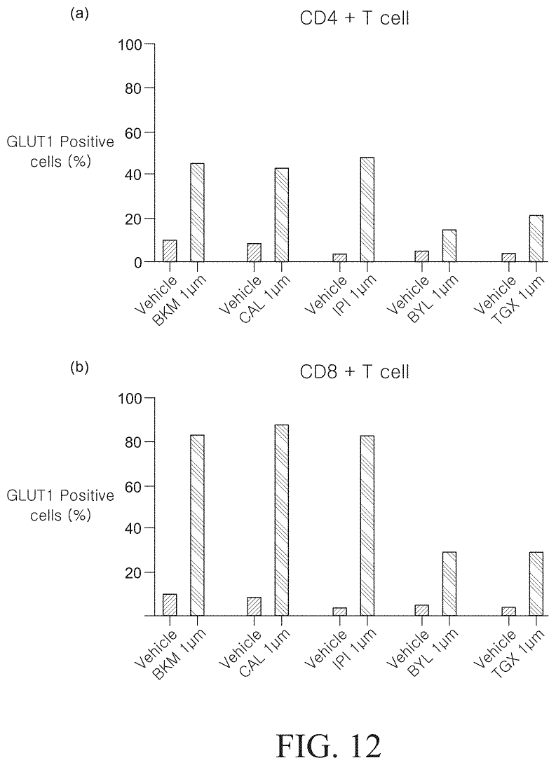

Hereinafter, with reference to FIG. 11 to FIG. 12, a method for enhancing immune function of an immune cell according to an example of the present disclosure and an immune cell line produced using the method will be described in detail.

First, referring to FIG. 11, a procedure for a method for enhancing the immune function of immune cells according to an example of the present disclosure is shown. The method may include treating the immune cell with a PI3K inhibitor (S110) and culturing the immune cell treated with the PI3K inhibitor (S120).

More specifically, in the step of treating the immune cell with the PI3K inhibitor (S110), the immune cell isolated from a biological sample is treated with the PI3K inhibitor to activate the AKT/mTOR downstream signal ex vivo. Accordingly, a level of AKT as a downstream signal from PI3K in the PI3K signaling system for immune cells is reduced.

In this connection, the injected PI3K inhibitor may be applied at a concentration of 0.5 to 2 μM, but is not limited thereto. The concentration may vary depending on the type and condition of immune cells. However, preferably, the concentration of the PI3K inhibitor may be 1 μM. Furthermore, the PI3K inhibitor may be injected 1 to 2 times per day (1 to 2/day). The immune cells may be treated continuously throughout a culture period.

The term “immune cell” as used herein refers to a cell capable of acting to defend a host from invading bacteria, viruses, toxins, tumors and other non-host materials, and may include dendritic cells, macrophage cells, B cells, T cells, NK cells, but is not limited thereto. In this connection, in one implementation, the T cell includes at least one selected from a group consisting of a helper T cell, a cytotoxic T cell, a memory T cell, an effect T cell, a regulatory T cell, a natural killer T cell, a mucosa-related constant T cell, an alpha-beta T cell, and a gamma-delta T cell. However, the present disclosure is not limited thereto.

Furthermore, the above-mentioned immune cells may include a TCR and/or CAR capable of recognizing an antigen of a target cell, a nucleic acid encoding the TCR and CAR, and a vector containing the aforementioned nucleic acid.

For example, the nucleic acid capable of encoding the TCR and CAR may be introduced into immune cells via the vector. In this connection, the vector may mean a nucleic acid molecule capable of transferring or transporting a nucleic acid molecule. The vector may contain sequences indicating autonomous replication in the cell or may contain sufficient sequences to enable integration into host cell DNA. The vector may include, but is not limited to, RNA plasmid, DNA plasmid, transposon, cosmid, bacterial artificial chromosome, and virus.

The immune cell may be isolated from a variety of biological samples containing mononuclear cells. The sample may include blood, plasma, lymph nodes, spleen, thymus, and bone marrow but is not limited thereto. The sample may include any sample from which the immune cell may be isolated.

Further, as used herein, the term “PI3K inhibitor” may mean a nucleic acid, peptide, compound or small organic molecule that binds to at least one of activators of PI3K and inhibits PI3K signaling. The PI3K inhibitor may selectively inhibit PI3K subunit modulators (p110α, β, γ, and δ) or may inhibit all of PI3K subtypes.

However, in the method for enhancing immune function of immune cells according to an example of the present disclosure, the inhibitor capable of enhancing the immune function of the immune cell may be a PI3Kδ inhibitor capable of inhibiting p110δ.

More specifically, referring to (a) of FIG. 12, the results of GLUT1 expression according to the type of PI3K inhibitor in the helper T cell are shown. In this connection, the PI3K inhibitor may include BKM 120 that may inhibit all subtypes, CAL-101 that may inhibit only p110δ, IPI-145 capable of inhibiting p110γ and P110δ, BYLT719 capable of inhibiting only p110α, and TGX-211 capable of inhibiting only p110β.

In all the groups treated with the PI3K inhibitor, GLUT1 expressions are higher than that in the control (vehicle) regardless of the type of PI3K inhibitor. Furthermore, BKM 120, CAL-101, and IPI-145 inhibitors exhibit higher GLUT1 expression levels than GLUT1 expression levels which BYLT719 and TGX-211 inhibitors exhibit. That is, all of the aforementioned BKM 120, CAL-101, and IPI-145 are inhibitors capable of inhibiting p110δ. Thus, the PI3Kδ inhibitor may increase GLUT1 expression more effectively.

Referring to (b) of FIG. 12, the results of GLUT1 expression according to the type of PI3K inhibitor in cytotoxic T cells are shown.

In all the groups treated with the PI3K inhibitor, GLUT1 expressions are higher than that in the control (vehicle) regardless of the type of PI3K inhibitor. Furthermore, BKM 120, CAL-101, and IPI-145 inhibitors exhibit higher GLUT1 expression levels than those of BYLT719 and TGX-211 inhibitors. That is, in the cytotoxic T cell as in the helper T cell, the PI3Kδ inhibitor may increase the GLUT1 expression more effectively.

Thus, the PI3K inhibitor to allow the AKT level to rebound via inhibition of the PI3K signal and to become a stronger signal to enhance glycolysis may be PI3Kδ, but may not be limited thereto. However, the PI3K inhibitor that may heterogeneously increase the metabolic action of immune cells may be preferably a PI3Kδ inhibitor.

Furthermore, the PI3Kδ inhibitor capable of inhibiting the PI3K subunit modulator p110δ includes at least one selected from a group consisting of selective PI3Kδ inhibitor 1(compound 7n), idelalisib/CAL-101, GS-1101, IC-87114, ZSTK474, PIK-294, PIK-293, leniolisib/CDZ173, GSK2292767, nemiralisib/GSK2269557, acalisib/GS-9820, PI-3065, AMG319, seletalisib/UCB-5857, umbralisib/TGR-1202, PIK-3, IPI-3063, XL147 analogue, PIK-90, taselisib/GDC0032, SRX3207, pilaralisib/XL147, pictilisib/GDC-0941, AZD8835, LY294002, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34inhibitor 1(compound 19), samotlisib/LY3023414, MTX-211, autophinib, VPS34-IN1, GNE-317, GDC-0084, bimiralisib/PQR309, SAR405, PF-49892416, duvelisib/IPI-145, TG100-115, tenalisib/RP6530, and AZD8186. However, the present disclosure is not limited thereto.

Further, the PI3Kα inhibitor capable of inhibiting the PI3K subunit modulator p110α includes at least one selected from a group consisting of XL147 analogue, PIK-90, taselisib/GDC0032, SRX3207, pilaralisib/XL147, alpilisib/BYL719, PIK-71 HCL, A66, gedatolisib/PKI-587, fimepinostat/CUDC-907, CH5132799, serabelisib/TAK-117, 2-D08, HS-173, GDC-0326, GNE-477, pictilisib/GDC-0941, AZD8835, LY294002, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34inhibitor 1(compound 19), samotlisib/LY3023414, MTX-211, autophinib, VPS34-IN1, GNE-317, GDC-0084, bimiralisib/PQR309, SAR405, PF-49892416, BGT226 maleate, and SF2523. However, the present disclosure is not limited thereto.

Further, the PI3Kβ inhibitor capable of inhibiting the PI3K subunit modulator p110β includes at least one selected from a group consisting of TGX-221, AZD6482, GSK2636771, LY294002, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34inhibitor 1(compound 19), samotlisib/LY3023414, MTX-211, autophinib, VPS34-IN1, GNE-317, GDC-0084, bimiralisib/PQR309, SAR405, PF-49892416, BGT226 maleate, and AZD8186. However, the present disclosure is not limited thereto.

Further, the PI3Kγ inhibitor capable of inhibiting the PI3K subunit modulator p110γ includes at least one selected from a group consisting AS-605240, 3-methyladenine/3-MA, voxtalisib/XL765, AS-252424, AS-604850, CAY10505, CZC24832, IPI-549, dactolisib/BEZ235, buparlisib/BKM120, PI-103, NU7441(KU-57788), wortmannin, omipalisib/GSK2126458, PF-04691502, apitolisib/GDC-0980, GSK1059615, copanlisib/BAY80-6946, PKI-402, TG100713, VS-5584(SB2343), VVPS34 inhibitor 1(compound 19), samotlisib/LY3023414, MTX-211, autophinib, VPS34-IN1, GNE-317, GDC-0084, bimiralisib/PQR309, SAR405, PF-49892416, duvelisib/IPI-145, TG100-115, tenalisib/RP6530, BGT226 maleate, and SF2523. However, the present disclosure is not limited thereto.

Furthermore, in the step (S110) in which the PI3K inhibitor is applied to the immune cell, the immune cell may be treated with CD3 and CD28 antibodies together with the PI3K inhibitor in order to activate the immune cell in vitro. For example, immune cells may be activated by exposure thereof to CD3 and CD28 antibodies immobilized on the surface of the bead. However, the method for the activation of immune cells is not limited to the above-mentioned antibody treatment method and may include any activation method for proliferation and maintenance of T cells. For example, a method of treating the cell with combination of CD3 antibody and interleukin-2, and a method of treating the cell with artificial antigen presenting cell containing CD3 antibody and 4-IBBL protein on the surface of fine particles may be used.

Then, the step of culturing the immune cells treated with the PI3K inhibitor (S120) may be performed for 3 to 4 weeks. In this connection, the immune cells treated with the PI3K inhibitor may be cultured in vitro in a mixed medium for growth and proliferation of cells containing essential elements for the growth and proliferation of cells such as sugars, amino acids, various nutrients, serum, growth factors, and minerals. The mixed medium may include, but is not limited to, Eagle's Minimum Essential Medium (Eagle's MEM), Dulbecco's modified Eagle's medium (DMEM), Ham's F 12, SF 12, RPMI 1640, and the like. Other substances may be contained therein.

Furthermore, in the step of culturing the immune cell treated with the PI3K inhibitor (S120), the AKT level which has been reduced via treatment with the PI3K inhibitor rebounds to increase. More specifically, due to the treatment of the PI3K inhibitor, the AKT level is increased again to a level before the PI3K treatment in 20 hours after the start of the culture. Thereafter, the AKT expression level is continuously increased by 48 hours after the start of culture and thus is higher by 3 times or greater than the AKT expression level at the start of culture. That is, when the immune cell is treated with the PI3K inhibitor and then the immune cell is continuously cultured, the AKT expression level may rebound and increase.

Thus, in accordance with the method for enhancing the immune function of the immune cell according to an example of the present disclosure, in the above-described process, the AKT expression in the immune cell is reduced, and thus the signal due to the decrease in the AKT expression may activate mTORC1. Then, the activated mTORC1 as a feedback signal may increase the expression of AKT by re-stimulating the AKT which has been reduced. Furthermore, the increased AKT level acts as a stronger signal than before the cell is treated with the PI3K inhibitor, thereby to heterogeneously increase the expression of GLUT1. Furthermore, glycolysis may be performed more effectively via the increased GLUT1, and thus more energy may be used and stored via the active glycolysis. Thus, the immune function of immune cells may be improved.

Then, in order to identify the improved immune function of the immune cell, the method for enhancing the immune function of the immune cell according to an example of the present disclosure may further include, after the step of culturing the immune cells treated with the PI3K inhibitor (S120), co-culturing the cultured immune cell with the tumor cell, and evaluating the percentage of tumor cells killed (apoptosis) by the immune cell, although the co-culturing and evaluating steps are not shown in FIG. 11

First, the step of co-culturing the immune cells with tumor cells may be carried out in at least one condition selected from normal culture conditions, hypoxic culture conditions, and low glucose culture conditions.

For example, cancel cells in the body may cause poor conditions, i.e., tumor microenvironment, via specific metabolic mutations, and evade attack from immune cells. The characteristics of the tumor microenvironment may include depletion of nutrition such as glucose and amino acid, high acidity, and hypoxia due to lactic acid accumulation.

Thus, in order to identify the enhanced immune function of the immune cell line as produced using the method for enhancing the immune function of immune cells according to an example of the present disclosure, the co-culturing may be performed in hypoxic culture conditions and low glucose culture conditions similar to the tumor microenvironment. However, the culture conditions that may be provided are not limited thereto but may include all culture conditions in which various conditions in the body may be simulated.

In one implementation, the tumor cell includes at least one selected from a group consisting of lung-cancer, non-small cell lung-cancer, pancreatic cancer, stomach cancer, liver cancer, breast cancer, cervical cancer, thyroid cancer, parathyroid cancer, prostate cancer, gallbladder cancer, biliary tract cancer, non-Hodgkin's lymphoma, Hodgkin's lymphoma, blood cancer, bladder cancer, kidney cancer, ovarian cancer, melanoma, colon cancer, bone cancer, skin cancer, head cancer, uterine cancer, rectal cancer, brain tumor, anal anus cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, urinary tract cancer, renal cell carcinoma, renal pelvic carcinoma, CNS (central nervous system) tumor, primary CNS lymphoma, spinal tumor, brainstem glioma and pituitary adenoma. However, the present disclosure is not limited thereto.

Then, the step of evaluating the percentage of the tumor cells as killed (apoptosis) by the immune cells may be carried out via fluorescence analysis using staining substance capable of distinguishing dead cells such as propidium iodine (PI) and annexin V. However, the present disclosure is not limited thereto. Various methods such as separation methods, electrochemical characteristics-based analysis, mass spectrometry, and the like may be used.

Accordingly, the percentage of the tumor cells killed by the immune cell lines produced using the method for enhancing immune function of immune cells according to an example of the present disclosure is in a range of 60 to 65% in normal culture conditions, is in a range of 45 to 50% in hypoxic culture conditions, and is in a range of 30 to 40% in low glucose culture conditions. Furthermore, in normal culture conditions, the percentage of the tumor cells killed using the present immune cell line is 2.5 times greater than that of conventional immune cells. Furthermore, in hypoxic culture conditions, the percentage of the tumor cells killed using the present immune cell line is 2.2 times greater than that of conventional immune cells. Furthermore, in low glucose culture conditions, the percentage of the tumor cells killed using the present immune cell line is 1.5 times greater than that of using conventional immune cells.

Accordingly, according to an example of the present disclosure, the method for enhancing the immune function of an immune cell and the immune cell line produced using the method may improve immune function capable of killing tumor cells, even in poor tumor microenvironment, compared to the conventional immune cells, and thus may have more effective anti-cancer effects.

Furthermore, the immune cell line produced using the method for enhancing the immune function of the immune cell according to an example of the present disclosure has the immune function such as the more effective anti-cancer effect, as described above and thus may be used as a pharmaceutical composition. For example, the immune cell line produced using the method for enhancing the immune function of an immune cell according to an example of the present disclosure may be used as a pharmaceutical composition, that is, an immunomodulatory cell therapeutic agent. In this connection, the immunomodulatory cell therapeutic agent means a pharmaceutical composition used for the purpose of treating a disease via activating an immune response in the body using immune cells.

Further, when the immune cell line produced using the method for enhancing the immune function of an immune cell according to an example of the present disclosure is used as the pharmaceutical composition, the composition may further contain at least one pharmaceutical active agent selected from cytokines, growth factors, hormones, small molecules, chemotherapeutic agents, pro-drugs, and antibodies. However, the present disclosure is limited thereto.

Further, when the immune cell line produced using the method for enhancing the immune function of an immune cell according to an example of the present disclosure is used as the pharmaceutical composition, the composition may further contain pharmaceutically acceptable carriers, diluents, adjuvants, excipients, preservatives, suspending agents, stabilizers, surfactants, emulsifiers, wetting agents, sweeteners, solvents, etc. However, the present disclosure is not limited thereto.

Accordingly, the pharmaceutical composition containing the immune cell line produced using the method for enhancing the immune function of an immune cell according to an example of the present disclosure and the additive agents may be administered to an individual who is immunocompromised, or who requires immune enhancement, thereby to treat and prevent the disease.

In this connection, as used herein, the term “pharmaceutical composition” means a pharmaceutically and physiologically acceptable formulated composition to be administered to a cell or animal, alone or in combination with one or more other forms of therapy.

### EXAMPLE 2

### Method for Enhancing Immune Function of Immune Cell and Anti-Cancer Effect of Immune Cell Line Produced Using the Method

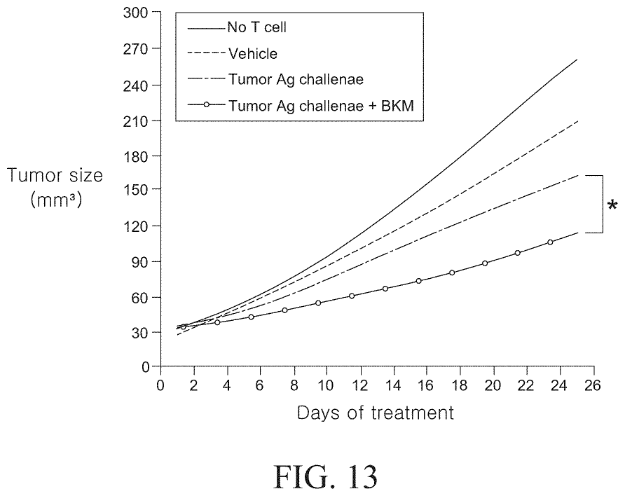

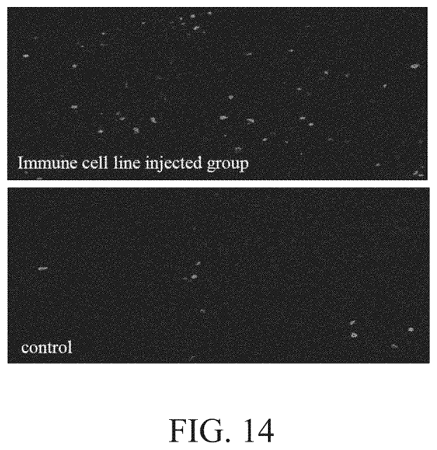

Hereinafter, with reference to FIG. 13 to FIG. 14, the anti-cancer effect of the immune cell line produced by the above-described method will be described in detail.

First, referring to FIG. 13, results of anti-cancer effects of the immune cell lines according to an example of the present disclosure are shown. In this connection, prior to the anti-cancer effect validation experiment, lung-cancer cell line LLC2 is transplanted into a nude mouse lacking endogenous T cells. Then, the immune cell line according to an example of the present disclosure is injected into the nude mouse, and the tumor size is measured to verify the anti-cancer effect.

First, when there is no injection (No T cell), the size of the tumor increases rapidly as the treatment day increases, and thus the tumor size on the 26th day is 240 to 270 mm³. The tumor size on the 26th day when the cell line is treated with the vehicle is found to be 190 to 220 mm³. Further, the tumor size on the 26th day when the cell line is treated with an antigen challenge to which the PI3K inhibiter is not added is 150 to 170 mm³. The tumor size on the 26th day when the immune cell line is treated with an antigen challenge to which the PI3K inhibiter is added is 110 to 120 mm³ and thus is significantly smaller than those of the other treatment groups as described above (p<0.05).

In other words, injecting the immune cell line according to an example of the present disclosure may improve anti-cancer effect capable of effectively reducing the tumor size.

Next, referring to FIG. 14, the infiltration of the immune cells into the tumor, that is, the results for the tumor, when the immune cell line according to an example of the present disclosure is used, is shown. In this connection, DAPI is used to stain nucleus (blue) of the tumor-infiltrating lymphocyte which is in contrast to CD3 expression (green).

More specifically, when injecting an immune cell line according to an example of the present disclosure, the amount of the tumor-infiltrating lymphocytes is larger and CD3 expression level is higher than those in the control. This may mean that the immune cells capable of penetrating the tumor and performing the immune response may increase due to injection of the immune cell line according to an example of the present disclosure.

Although the embodiments of the present disclosure have been described in more detail with reference to the accompanying drawings, the present disclosure is not necessarily limited to these embodiments, and various modifications may be carried out without departing from the technical idea of the present disclosure. Therefore, the embodiments disclosed in the present disclosure are not intended to limit the technical idea of the present disclosure. The scope of the technical idea of the present disclosure is not limited by these embodiments. Therefore, it should be understood that the embodiments described above are illustrative in all respects and not restrictive. The scope of protection of the present disclosure should be interpreted by the claims below. All technical ideas within the equivalent scope should be interpreted as being included in the scope of the present disclosure.

What is claimed is:

1. A method for enhancing immune function of an immune cell, the method comprising:
treating immune cells with a PI3K inhibitor to activate AKT/mTOR downstream signal ex vivo; and
culturing the immune cells treated with the PI3K inhibitor such that a lowered AKT level rebounds.

2. The method of claim 1, further comprising treating the immune cells with CD3 and CD28 antibodies to activate the immune cell.

3. The method of claim 1, further comprising:
co-culturing the cultured immune cells together with tumor cells; and
evaluating a percentage of an amount of tumor cells killed by the immune cells.

4. The method of claim 3, wherein the tumor cell includes at least one selected from a group consisting of lung-cancer, non-small cell lung-cancer, pancreatic cancer, stomach cancer, liver cancer, breast cancer, cervical cancer, thyroid cancer, parathyroid cancer, prostate cancer, gallbladder cancer, biliary tract cancer, non-Hodgkin's lymphoma, Hodgkin's lymphoma, blood cancer, bladder cancer, kidney cancer, ovarian cancer, melanoma, colon cancer, bone cancer, skin cancer, head cancer, uterine cancer, rectal cancer, brain tumor, anal anus cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, urinary tract cancer, renal cell carcinoma, renal pelvic carcinoma, CNS (central nervous system) tumor, primary CNS lymphoma, spinal tumor, brainstem glioma and pituitary adenoma.

5. The method of claim 3, wherein the co-culturing is performed in at least one of normal, hypoxic, and low glucose culture conditions.

6. The method of claim 1, further comprising isolating the immune cells from a biological sample.

7. The method of claim 6, wherein the biological sample includes at least one selected from a group consisting of blood, plasma, lymph nodes, spleen, thymus, and bone marrow.

8. The method of claim 1, wherein the immune cell includes at least one selected from a group consisting of a dendritic cell, a macrophage cell, a B cell, a T cell, and a NK cell.

9. The method of claim 8, wherein the T cell includes at least one selected from a group consisting of a helper T cell, a cytotoxic T cell, a memory T cell, an effect T cell, a regulatory T cell, a natural killer T cell, a mucosa-related constant T cell, an alpha-beta T cell, and a gamma-delta T cell.

10. The method of claim 9, wherein the T cell contains TCR or CAR capable of recognizing an antigen of a target cell; a nucleic acid encoding the TCR or CAR; and a vector containing the nucleic acid.

11. The method of claim 9, wherein the NK cell contains TCR or CAR capable of recognizing an antigen of a target cell;
a nucleic acid encoding the TCR or CAR, and a vector containing the nucleic acid.

12. The method of claim 1, wherein the immune cells are treated with the PI3K inhibitor once or twice a day.

13. The method of claim 1, wherein the PI3K inhibitor has a concentration of 0.5 to 1 μM.

14. The method of claim 1, wherein the PI3K inhibitor includes at least one selected from a group consisting of PI3Kα, PI3Kβ, PI3Kγ, and PI3Kδ inhibitors.

15. The method of claim 14, wherein the PI3K inhibitor includes PI3Kδ inhibitor.

16. An immune cell line, wherein the immune cell line is treated with a PI3K inhibitor, wherein a percentage of immune cells expressing GLUT1 among total immune cells for the immune cell line is equal to or greater than 40%.

17. The immune cell line of claim 16, wherein the GLUT1 expression level of the immune cell line is 2 to 3 times greater than a GLUT1 expression level of a control non-treated with the PI3K inhibitor.

18. The immune cell line of claim 16, wherein the immune cell line includes at least one selected from a group consisting of a helper T cell, a cytotoxic T cell, a memory T cell, an effect T cell, a regulatory T cell, a natural killer T cell, a mucosa-related constant T cell, an alpha-beta T cell, and a gamma-delta T cell.

19. The immune cell line of claim 18, wherein when each of the helper T cell and the cytotoxic T cell is treated with the PI3K inhibitor, a lowered AKT level is rebounded to increase to a level 1.5 times or greater than an AKT level before the PI3K inhibitor treatment, for 48 hours.

20. The immune cell line of claim 19, wherein the cytotoxic T cells are treated with the PI3K inhibitor, wherein a percentage of the cytotoxic T cells expressing GLUT1 among total cytotoxic T cells for the immune cell line is equal to or greater than 80%.
